(19) United States
(12) Patent Application Publication
(10) Pub. No.: US 2007/0065414 A1
(43) Pub. Date: Mar. 22, 2007

(54) Enhanced delivery of cells

(75) Inventors: Toby Freyman, Waltham, MA (US); Maria Palasis, Wellesley, MA (US); Wendy Naimark, Cambridge, MA (US)

Correspondence Address: KENYON & KENYON LLP, 1500 K STREET N.W., SUITE 700, WASHINGTON, DC, 20005, US

(73) Assignee: Boston Scientific Scimed, Inc.

(21) Appl. No.: 11/227,278

(22) Filed: Sep. 16, 2005

(51) Int. Cl. A61K 35/14; C12N 5/08; A61K 38/20

(52) U.S. Cl. 424/93.7; 435/372; 424/85.2; 514/12; 514/45; 514/509; 514/419; 514/400; 514/573; 514/565

(57) ABSTRACT

The present invention provides a method comprising administering cells by infusion through a blood vessel and administering a vasodilator, a vascular enhancer, or both. In accordance with one embodiment, the delivery of stem cells to the myocardium by intracoronary infusion is improved by administration of one or more vasodilators and/or vascular permeability enhancers prior to or at the time of cell therapy.

### FIELD OF THE INVENTION

The present invention relates to enhanced delivery of cells through a blood vessel by administration of a vasodilator and/or a vascular permeability enhancer.

### BACKGROUND OF THE INVENTION

Congestive heart failure—the ineffective pumping of the heart caused by the loss or dysfunction of heart muscle cells—is a leading cause of death in the United States. A major cause of congestive heart failure is a heart attack, known medically as a myocardial infarction. Standard reperfusion therapies for restoring heart function include surgical revascularization with bypass operation, administration of clot-busting drugs, and/or interventional cardiology such as PTCA (percutaneous transluminal coronary angioplasty), balloon angioplasty, and stent implantation.

Stem cell therapy shows promise as a means to repair and/or replace the cells vital to heart health, particularly the cardiomyocytes which comprise the heart muscle and contract to pump blood, the vascular endothelial cells which form the inner lining of new blood vessels, and smooth muscle cells which form the walls of blood vessels. In vitro studies have shown that stem cells can be induced to develop into new cardiomyocytes and vascular endothelial cells. See Stem Cells: Scientific Progress and Future Research Directions, Chapter 9: Can Stem Cells Repair a Damaged Heart?, Department of Health and Human Services (2001), available at http://stemcells.nih.gov/info/scireport/chapter9.asp. Clinical studies have shown that intracoronary infusion of stem cells may beneficially affect postinfarction remodeling processes. Assmus et al., “Transplantation of Progenitor Cells and Regeneration Enhancement in Acute Myocardial Infarction,” Circulation 106:3009 (2002), available at http://circ.ahajournals.org/cgi/content/full/106/24/3009.

Intracoronary infusion allows local delivery of therapeutic formulations to tissue by infusion through a blood vessel. Intracoronary infusion is less invasive than treatments that require opening of the thoracic cage such as some standard reperfusion therapies and intramyocardial transplantation whereby cells are injected directly into the muscle. However, infusion of cells through a blood vessel may cause occlusion of the blood vessel, particularly for the infusion of large cells.

### SUMMARY OF THE INVENTION

In one embodiment, the present invention provides a method comprising administering cells by infusion through a blood vessel and administering a vasodilator, a vascular enhancer, or both. The cells may be stem cells. The vasodilator and/or the vascular permeability enhancer may be administered prior to administering the stem cells. The administration of the stem cells and the administration of the vasodilator and/or the vascular permeability enhancer may be performed by simultaneous intracoronary infusion. One embodiment is the simultaneous administration of mesenchymal stem cells and adenosine II by intracoronary infusion.

In one embodiment, the method further comprises infusing a saline pre-infusate, for example comprising plasma proteins, before administering the stem cells.

### DETAILED DESCRIPTION OF EMBODIMENTS OF THE INVENTION

In one embodiment, the present invention provides a method comprising administering stem cells by intracoronary infusion and administering a vasodilator, a vascular enhancer, or both.

The stem cells, the vasodilator, and/or the vascular permeability enhancer may be administered in any order or in multiple doses. Preferably, the vasodilator and/or the vascular permeability enhancer are administered close in time to the administration of the stem cells such that the vasodilator and/or the vascular permeability enhancer improve the delivery of the stem cells to the myocardial tissue. The vasodilator and/or the vascular permeability enhancer may be administered prior to and/or simultaneously with the delivery of the stem cells.

The vasodilator and/or the vascular permeability enhancer may be administered by any means known in the art, including, for example, intravenous administration and intracoronary administration. Preferably, the vasodilator and/or the vascular permeability enhancer are administered such they affect only the local environment. Thus, they are preferably administered at the site of stem cell administration. Accordingly, in a preferred embodiment, the stem cells, the vasodilator, and/or the vascular permeability enhancer are all administered by intracoronary infusion.

In one embodiment, the stem cells are administered by intracoronary infusion, which generally encompasses infusing the stem cells into the vascular tree of coronary arteries, arterioles, and capillaries. Preferably, the infusion is performed into the infarct artery. Intracoronary infusion of stem cells is generally performed by using a catheter to deliver the stem cells into the blood vessel. For instance, a balloon catheter is advanced into a previously implanted stent. To allow for adhesion and potential transmigration of the infused cells through the endothelium, the balloon is inflated with low pressure to completely block blood for about 3 minutes while the stem cell suspension is infused distally to the occluding balloon through the central port of the balloon catheter. The method may be repeated more than once. Multiple infusions may be interrupted by short periods of about 3 minutes of reflow by deflating the balloon to minimize extensive ischemia. See Assmus et al., “Transplantation of Progenitor Cells and Regeneration Enhancement in Acute Myocardial Infarction,” Circulation 106:3009 (2002), available at http://circ.ahajournals.org/cgi/content/full/106/24/3009. The stem cells may be harvested and prepared for infusion by any means known in the art.

The present invention also provides a method of administering stem cells through blood vessels other than the vascular tree of coronary arteries, arterioles, and capillaries. The administration of the stem cells, the vasodilator, and/or the vascular permeability enhancer can be performed by infusion into a blood vessel generally. The blood vessel may lead, for example, to the heart, brain, liver, kidney, pancreas, or lung.

Stem cells include, but are not limited to, embryonic stem cells such as early embryonic stem cells and blastocyst embryonic stem cells; fetal stem cells; umbilical cord stem cells; and adult stem cells such as mesenchymal stem cells, hematopoietic stem cells, endothelial stem cells, peripheral blood stem cells, and multipotent somatic stem cells. In one embodiment, mesenchymal stem cells are preferred.

In order to prevent or reduce the rejection of transplanted cells, the administered stem cells in some embodiments are preferably autologous. The autologous stem cells may be harvested from any source, for example, from bone marrow or peripheral blood.

The method of the present invention may also be useful for delivery of cells for gene therapy. In this embodiment, allogenic rather than autologous cells may be preferred.

A vasodilator enhances the ability of administered stem cells to traverse through the blood vessels and capillaries to the desired site of transplantation, such as the myocardial tissue. Vessel dilation improves delivery of the stem cells and reduces the risk of myocardial ischemia secondary to capillary occlusion. The vasodilator may also reduce the infarct size. Vasodilators useful for the present invention include, but are not limited to, endogenous/metabolic vasodilators such as lactic acid, adenosine triphosphate, adenosine diphosphate, adenosine monophosphate, adenosine, adenosine II, nitric oxide, hemoxygenase, VEGF, and agents causing hypercapnia, hypoxia/hypoxemia, or hyperemia; phosphodiesterase inhibitors such as dipyridamole and sildenafil; sympathetic activity inhibitors such as clonidine and methyldopa; smooth muscle relaxants such as papaverine, hydralazine, dihydralazine, and nitroprusside; beta receptor agonists such as dopamine, dobutamine, arbutamine, albuterol, salmeterol, and isoproterenol; alpha receptor antagonists such as doxazosin, terazosin, and prazosin; organic nitrates, such as glyceryl trinitrate, isosorbide dinitrate, and isosorbide mononitrate; angiotensin converting enzyme (ACE) inhibitors such as benazepril, captopril, enalapril, fosinopril, lisinopril, quinapril, and ramipril; angiotensin II antagonists (or ATI receptor antagonists) such as valsartane, losartan, and candesartan; calcium channel blockers such as amlodipine, nicardipine, nimodipine, felodipine, isradipine, diltiazem, verapamil, and nifedipine; prostaglandins such as alprostadil; and endothelium-dependent vasodilators; and also the vasodilators minoxidil, nitroglycerin, bosentan, eporprostenol, and treprostinil. In one embodiment, the vasodilator is preferably adenosine II, hydralazine, minoxidil, nitroglycerin, an angiotensin converting enzyme inhibitor, bosentan, eporporstenol, treprostinil, or a calcium channel blocker, and most preferably adenosine II.

Because the required vasodilatation may need only to be short lasting, adenosine is a particularly useful vasodilator. Adenosine is an endogenous substance, and it has a very short-lasting action as evidenced by a blood pool half-life of only a few seconds. Vasodilatation will accordingly be most intense at the site of administration, since the drug will tend to reach more distal tissues in less than pharmacologically active concentrations.

A vascular permeability enhancer enhances the ability of the administered stem cells to pass through the vessel wall to the desired site of transplantation, such as the myocardial tissue. Since the cells reach the myocardial tissue via the vascular/capillary bed, agents which enhance vascular permeability are expected to also enhance the levels of stem cells which reach the myocardial tissue. Vascular permeability enhancers useful for the present invention include, but are not limited to, serotonin, bradykinin, platelet-activating factor, prostaglandin E₁, histamine, vascular endothelium growth factor, zona occludens toxin, interleukin-2, plasma kinins, L-N-monomethyl arginine, L-N-nitro-arginine methyl ester, alcohol such as ethanol and isopropanol, polyethylene glycols, fatty acid molecules with 10 to 20 carbon rings and certain mono-, di-, and triglycerides of fatty acids.

The delivery of cells to the desired site of transplantation may also be enhanced by administering a saline pre-infusate before administering the stem cells. Infusing saline before infusing the stem cells increases the hydrostatic and/or osmotic pressure, thereby driving the stem cells into the interstitium. A saline pre-infusate that contains plasma proteins may further enhance interstitial transport.

The method of the present invention may be useful to repair or replace damaged tissue, especially heart tissue. Without being bound by theory, it is believed that stem cells replace or repair damaged heart tissue by cell-associated myocardial regeneration and neovascularization. Accordingly, preferred subjects of administration for the present invention include subjects, particularly human subjects, suffering from damaged or diseased heart tissue. An especially preferred subject is a human who has suffered an acute myocardial infarction (AMI). Preferred subjects of administration also include, but are not limited to subjects, particularly human subjects, suffering from damaged or diseased tissue of the brain, liver, kidney, pancreas, or lung. Other preferred subjects include candidates for gene therapy.

Having thus described the invention with reference to particular preferred embodiments, those in the art can appreciate modifications to the invention that do not depart from the spirit and scope of the invention as disclosed in the specification and defined by the following claims. The embodiments are set forth to aid in understanding the invention but are not intended to, and should not be construed to, limit its scope in any way.

What is claimed is:

1. A method of delivering cells through a blood vessel comprising:
a. administering cells by infusion through the blood vessel; and
b. administering through the blood vessel a vasodilator, a vascular permeability enhancer, or both.

2. The method of claim 1, wherein the vasodilator, the vascular permeability enhancer, or both are administered by infusion.

3. The method of claim 1, wherein the vasodilator, the vascular permeability enhancer, or both are administered prior to administering the cells.

4. The method of claim 1, wherein the vasodilator, the vascular permeability enhancer, or both are administered simultaneously with the cells.

5. The method of claim 1, wherein the cells are stem cells.

6. The method of claim 5, wherein the stem cells are embryonic stem cells, adult stem cells, mesenchymal stem cells, hematopoietic stem cells, endothelial stem cells, peripheral blood stem cells, or multipotent somatic stem cells.

7. The method of claim 5, wherein the stem cells are autologous.

8. The method of claim 1, wherein the vasodilator is adenosine II, hydralazine, minoxidil, nitroglycerin, an angiotensin converting enzyme inhibitor, bosentan, eporporstenol, treprostinil, or a calcium channel blocker.

9. The method of claim 1, wherein the vascular permeability enhancer is serotonin, bradykinin, platelet-activating factor, prostaglandin E₁, histamine, vascular endothelium growth factor, zona occludens toxin, interleukin-2, plasma kinins, L-N-monomethyl arginine, or L-N-nitro-arginine methyl ester.

10. The method of claim 1, further comprising infusing a saline pre-infusate before administering the stem cells.

11. The method of claim 10, wherein the saline pre-infusate comprises plasma proteins.

12. The method of claim 1, wherein the blood vessel leads to the heart, brain, liver, kidney, pancreas, or lung.

13. A method of repairing or replacing heart tissue comprising administering a vasodilator and stem cells by intracoronary infusion.

14. The method of claim 13, wherein the vasodilator is administered prior to administering the stem cells.

15. The method of claim 13, wherein the vasodilator is administered simultaneously with the stem cells.

16. The method of claim 13, wherein the stem cells are mesenchymal stem cells.

17. A method of repairing or replacing heart tissue comprising administering a vascular permeability enhancer and stem cells by intracoronary infusion.

18. The method of claim 17, wherein the vascular permeability enhancer is administered prior to administering the stem cells.

19. The method of claim 17, wherein the vascular permeability enhancer is administered simultaneously with the stem cells.

20. The method of claim 17, wherein the stem cells are mesenchymal stem cells.
